(19) United States
(12) Patent Application Publication
(10) Pub. No.: US 2020/0325483 A1
(43) Pub. Date: Oct. 15, 2020

(54) SAFE DELIVERY OF CRISPR AND OTHER GENE THERAPIES TO LARGE FRACTIONS OF SOMATIC CELLS IN HUMANS AND ANIMALS

(71) Applicant: Brian P. Hanley, Davis, CA (US)

(72) Inventor: Brian P. Hanley, Davis, CA (US)

(21) Appl. No.: 16/097,886

(22) PCT Filed: May 12, 2017
(86) PCT No.: PCT/US2017/032470

§ 371 Date: Oct. 31, 2018

Related U.S. Application Data
(60) Provisional application No. 62/335,561, filed on May 12, 2016.

(51) Int. Cl. C12N 15/63; C12N 15/10; A61K 31/436; A61K 31/7105

(52) U.S. Cl. CPC C12N 15/635; C12N 15/102; A61K 45/06; A61K 31/7105; A61K 31/436

(57) ABSTRACT

A method for making it possible to deliver nucleic acid sequences to a large fraction of, or even 99.9% and more of the cells in a human body without near certainty of killing the recipient. It can be applied to safely deliver any gene therapy. This invention comprises a set of known compounds, many of them already approved, combined in novel ways to prevent immune system reaction to levels of delivery vehicle (capsid or synthetic carrier) introduced into the body that can be 5 or more orders of magnitude higher than has been demonstrated to cause human death. When used in concert with the disclosed CRISPR expression control method, this method can improve expression and allow better control over the gene therapy's target activity.

### RELATED APPLICATIONS

This application claims priority under 35 U.S.C § 371 to PCT application No. PCT/US2017/032470 filed May 12, 2017, which is based on and claims priority to United States U.S. Patent Application 62/335,561, filed May 12, 2016.

### BACKGROUND OF THE DISCLOSURE

### Technical Field of the Disclosure

This invention is in the field of gene therapies and enabling technologies for same.

### Description of the Related Art

The invention requires understanding of the mechanisms of sepsis and stimulation of toll-like receptors (TLRs). It also requires basic understanding of CRISPR, what a gene therapy is and what a CHYSEL system linker is.

### Immune System Vulnerability to High-Dose Stimulation Sepsis Like Syndrome.

The cause of sepsis is commonly thought to be infection. However, technically speaking, sepsis is due to pathogen associated molecular patterns (PAMPs) that the immune system is sensitive to. These trigger toll like receptors (TLRs) c-type lectin receptors (CLRs) and cytoplasmic pattern recognition receptors (cPRRs) that respond to nucleic acid motifs. In addition, damage associated molecular patterns (DAMPs) from cellular debris also trigger inflammatory cytokines.

The root cause of sepsis can be PAMPs from bacteria, fungi or viruses, but it is usually triggered by broken down bacterial cell walls, technically referred to as endotoxin, or lipopolysaccharide (LPS). Thus, a standard laboratory model of sepsis is injection of sterile LPS. This can also duplicate multi-organ failure which is often triggered by DAMP overstimulation. In the present case, the triggering agent is virion capsids, most often those of adeno associated virus (AAV) strains or lentivirus (LV) strains. Additionally, there are cytosolic receptors for nucleic acids, and there is cross-talk between TLRs.

In mouse model the LD₅₀ of LPS depends on liver protein synthesis. Normal LPS LD₅₀ in 25 gram mice is approximately 150 micrograms (6 mg/kg), taking 35 hours to complete its course. However, in mice pretreated with β-galactosamine to inhibit liver protein synthesis, the mouse LD₅₀ is approximately 5 nanograms (200 ng/kg), 30,000 times less. By comparison, normal humans show reactions at 2-4 nanograms of LPS per kilogram. The human LD₅₀ for LPS is below 5 micrograms per kilogram, three orders of magnitude lower than mice. Humans are 6000 times larger yet die at doses only double the dose that kills a 25-gram mouse. If humans had the immune systems of mice, the LPS LD₅₀ would be around 900 milligrams for an average adult instead of 300 micrograms. Human LPS sensitivity is believed to be due to mutations in sialic acid binding Ig-like lectins (siglecs). Phenotypically, knockouts of siglecs produce hyper-reactive immune system components. The missing siglec-13 and inactivated siglec-17 that are active in other primates affects control over TLR4. TLR4 is the receptor for LPS. It is also the target for HMGB1, in DAMP signaling.

The fact that human immune system control over TLR4 hyper-stimulation has been knocked out should always be kept in mind when looking at study results from any animal model, including all non-human primates. Animals can easily tolerate immune system challenges that kill humans. Adeno Associated Virus (AAV) triggers different TLRs than endotoxin. There are different strains of AAV created for use in gene therapy delivery. In liver, Kupffer cells are sensitive to AAV2 through TLR2 and other cells are sensitive through TLR9. These responses can be blunted, but not eliminated, by blocking the receptors.

“TLR2 recognizes peptidoglycan from Gram-positive bacteria, zymosan from yeast cell walls, and glycophosphatidylinositol anchors from Trypanosoma cruzi. TLR2 can also recognize bacterial lipoproteins when dimerizing with TLR1 and mycoplasma lipoproteins when dimerizing with TLR6 . . . . TLR9 is activated by unmethylated CpG DNA motifs present in bacterial DNA and in viruses such as mouse cytomegalovirus and herpes simplex viruses.”

There are two major inflammatory pathways, the classical (canonical, direct) and alternative (noncanonical, indirect). The classical pathway stimulates through ‘myeloid differentiation primary response gene 88’ (MyD88), the alternative stimulates through ‘TIR-domain-containing adapter-inducing interferon-β’ (TRIF). Both hit ‘nuclear factor-κB’ (NFκB). TRIF stimulates IRF3, while MyD88 stimulates RF7, both of which causes production of Type I interferons.

Type I interferons in humans are: IFN-α (13 subtypes), IFN-β1 & IFN-β3, IFN-κ, IFN-ω1. There is only one type II interferon: IFN-γ.

The Toll-like receptor 9 (TLR9) receptor is triggered by cytosine-phosphate-guanine (CpG) oligodeoxynucleotide (ODN) or CpG oligodeoxynucleotides (CPG-ODNs). This receptor uses the TRIF system.

### Cell Years Concept

The concept of CRISPR (clustered regularly interspaced short palindromic repeats) cell years will be used throughout this document to illustrate the inventive features of the present method and structures. For this concept, it is assumed that there will never be a CRISPR system that is absolutely perfect. The probability of an off-target event that modifies DNA that is not intended, is some very low number times the expression level, multiplied by the number of cells times the number of years the CRISPR system is active in those cells.

E·P(e)≮S(e)·C·Y=P(R) Equation 1

Where: E=Expression level, P(e)=Probability of an off-target error, S(e)=Severity of the off-target error, C=number of cells, Y=number of years, and P(R)=probability of a potentially dangerous off target event. It is noted that a potentially dangerous off target event does not equal cancer.

Equation 1 may be used in a collative form that sums a set of expressions with differing values. For instance, E may vary based on the number of active cassettes within one cell, and also from the severity of off-target events. This equation 1 may be used as a guideline to generate values for risk estimation. However, note that radiation damage in cell culture led to Haldane's 1955 estimate of mutation doubling rate in humans of 0.05 gray whole body radiation Later studies showed the actual minimum radiation dose to be 2 gray, and probably 4 to 6 gray (e.g. mortal dose range). Mammals are quite good at destroying cells that have errors.

### CHYSEL System

The CHYSEL system was developed after recognizing that viruses have special amino acid sequences that allow them to split proteins after they have been translated. This innovation has made it possible to express equimolar quantities of different proteins from a single promoter. This can greatly simplify the expression.

### Problems to be Solved

There are two problems that obstruct the development of gene therapy in general and ‘Clustered Regularly Interspaced Short Palindromic Repeats’ (CRISPR) gene therapy in particular. These problems are: delivery of DNA or RNA to sufficient cells in a human body; and overlong expression of CRISPR.

### Problem 1—Delivery to Sufficient Numbers of Cells

A gene therapy patient, Jesse Gelsinger, died from a dose of 3.8×10¹³ adenovirus particles administered into his hepatic vein to deliver a gene therapy. Consequently, the normal limit for virions injected to accomplish gene therapy is 10¹². This invention is intended to prevent the syndrome that caused his death. The inventor was aware of this problem through graduate school, but like most others, accepted that it was difficult to solve and that the way to do it was to find alternative, less immunogenic delivery methods than adenovirus capsids, adeno-associated virus (AAV) capsids, lentivirus capsids, etc. There remain to this day many advocates for this strategy. However, the inventor in a previous disclosure, U.S. patent application Ser. No. 13/298,251, described treating HIV using gene therapy methods that delivered genes coding for anti-HIV antibodies that would act intra-cellularly. The delivery aspect of this patent focused him on the numbers involved in transfection in the human body. The inflammatory aspect of the immune system, and methods for controlling it, and the difference in response between synthetically produced sepsis and that which occurs in infection were clear in his mind. Unfortunately, very few people, including physicians and scientists who work with microbial methods for treatment of cancer, are aware of the uniqueness of human immunology relative to immune system hyper-activation. Consequently, most experts do not realize the scale of the gap between what is necessary and what is possible using existing methods. To address this requires that the immune system be controlled with modulators that inhibit or otherwise suppress or arrest the inflammatory cycle of the immune system.

As further framework for the present invention, it is noted there are 3.72×10¹³ cells give or take a few hundred billion in a human body. For comparison, a mouse has around 10¹² cells. Proportionally, a 20-gram mouse should have approximately 1.2×10¹⁰ cells. As a rule, when viral capsid packaging of gene therapy plasmids is used, approximately 1 in 1,000 capsids survive to transfect a cell.

The number of virions that a cell in a culture dish receives at different multiples of transfection (MOT), is described by the Poisson distribution:

P(k)=e^{-m}k/k! Equation 2

Where P(k) is the fraction of cells infected by k virus particles, and m is the MOT. The equation can be simplified to calculate the fraction of uninfected cells (k=0), cells with a single infection (k=1), and cells with multiple transfection (k>1):

P(0)=e^{-m} Equation 3

P(1)=me^{-m} Equation 4

P(K>0)=1−e^{-m} Equation 3.1 (implied above)

For purposes of the present disclosure, any cell with one or more transfections is fine. The overriding consideration is whether P(0) is sufficiently small that it is acceptable. So, using equation 3.1 for an MOT (m) of various values, the following table for a range of MOT from 10⁻¹⁰ to 10 may be prepared.

TABLE 1
MOT and P(0), per equation 3.1

| P(0) | MOT |
|---|---|
| 0.99999999990 | 1.0E−10 |
| 0.99999999900 | 1.0E−09 |
| 0.99999999000 | 1.0E−08 |
| 0.99999990000 | 1.0E−07 |
| 0.999999 | 1.0E−06 |
| 0.99999 | 1.0E−05 |
| 0.999900005 | 1.0E−04 |
| 0.9990005 | 1.0E−03 |
| 0.990049834 | 1.0E−02 |
| 0.904837418 | 1.0E−01 |
| 0.367879441 | 1 |
| 0.135335283 | 2 |
| 0.049787068 | 3 |
| 0.018315639 | 4 |
| 0.006737947 | 5 |
| 0.002478752 | 6 |
| 0.000911882 | 7 |
| 0.000335463 | 8 |
| 0.00012341 | 9 |
| 4.53999E−05 | 10 |

There are other methods of transfection. There are alternative synthetic carriers that enclose DNA to transfect cells, all of which have some level of toxicity, and all of which are applicable to the present invention.

Bare DNA plasmids can be administered intravenously to transfect cells, but to do so requires huge amounts and generation of over-pressure in the blood vessels locally, which is not practical in humans. That requires surgical intervention that is complex and potentially dangerous. Electroporation can be used, but it is practical in small regions.

### Problem 2—Expression of CRISPR

CRISPR is quite efficient at what it does, and there is the problem of off-target changes to DNA. There are better enzymes than the original CAS9 that have very low off-target fractions. The longer that the CAS9 enzyme is active, the greater the probability that it will make an off-target change with potential problems. Probably that potential is exceedingly low, but it is still a concern. The body has numbers of cells that dwarf the number of stars in the Milky Way galaxy by 100 times, and very low probability events become relatively likely. So, it is desirable to minimize the cell-years of activity by a particular CRISPR system. The assumption is it should take no more than a few days for near perfect modification to occur in any transfected cell.

The present embodiment overcomes the existing shortcomings in this area by accomplishing these objectives.

### SUMMARY OF THE DISCLOSURE

To minimize the limitations found in the prior art, and to minimize other limitations that will be apparent upon the reading of the specifications, the preferred embodiment of the present invention provides a method and related structures for the safe delivery of CRISPR and other gene therapies to large fractions of somatic cells in humans and animals.

This is primarily done through two key parts: delivery to sufficiently large numbers of somatic cells for significant organism transformation by transfecting 10% or more of somatic cells, and short-term expression of CRISPR. This invention uses multiple methods to achieve all three aspects so that these stumbling blocks to gene therapy can be solved. Additionally, the methods of delivery to large fractions of somatic cells is useful for other types of gene therapy.

First, using one or more medications, the immune system is suppressed until the viral capsid load injected is no longer a danger. Medications can be administered by any manner known to medicine, including, but not limited to, oral, intravenous, intralymphatic, subcutaneous, intraperitoneal, intramuscular, suppository, or transdermal. Second, plasmids known to be controlled within weeks are selected as the backbone for delivery of the CRISPR cassette, and/or promoters such as a eukaryote tetracycline/doxycycline inducer (tetR, TRE) that require the presence of tetracycline or a related drug (ex. Doxycycline) to activate the CRISPR gene cassette.

These and other advantages and features of the present invention are described with specificity so as to make the present invention understandable to one of ordinary skill in the art.

### DETAILED DESCRIPTION

In the following discussion that addresses a number of embodiments and applications of the present invention. It is to be understood that other embodiments may be utilized and changes may be made without departing from the scope of the present invention.

Various inventive features are described below that can each be used independently of one another or in combination with other features. However, any single inventive feature may not address any of the problems discussed above or only address one of the problems discussed above. Further, one or more of the problems discussed above may not be fully addressed by any of the features described below.

As used herein, the singular forms “a”, “an” and “the” include plural referents unless the context clearly dictates otherwise. “And” as used herein is interchangeably used with “or” unless expressly stated otherwise. As used herein, the term ‘about” means+/−5% of the recited parameter. All embodiments of any aspect of the invention can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “wherein”, “whereas”, “above,” and “below” and words of similar import, when used in this application, shall refer to this application as a whole and not to any particular portions of the application.

The description comprises two main parts. Part 1 makes it possible to inoculate a patient with an MOT sufficiently high. Part 2 designs expression cassette plasmids so that they will have a short and controlled expression life-span.

### Part 1: Delivery of Nucleic Acids to Sufficient Numbers of Cells

The first problem to address is how to effectuate a real MOT as high as 100 to be used in humans. Given the nature of the mechanisms involved, a real MOT of 100 would suggest that the formal MOT needs to be 10,000 to 100,000 in vivo. A set of immune system modulators are required to inhibit inflammation sufficiently as will be taught below.

TLR9 should be covered by a combination of nuclear factor κB (NFκB) and interferon regulatory factor 3 (IRF3) inhibitors. However, if this becomes a problem, there are specific drugs that inhibit these pathways, including but not limited to:
a. 3-[4-(6-(3-(dimethylamino)propoxy)benzo[d]oxazol-2-yl)phenoxy]-N,N-dimethylpropan-1-amine;
b. 6-[3-(pyrrolidin-1-yl)propoxy)-2-(4-(3-(pyrrolidin-1-1)propoxy)phenyl]benzo[d]oxazole;
c. and hydroxychloroquine.

The Nuclear factor-κB (NFκB) has drugs that act as inhibitors.

TABLE 2
NFκB inhibitors.

| | | | IκBα | caspase 3/7 | |
|---|---|---|---|---|---|
| | FDA | NFκB | phosphorylation | and | |
| Drug | approved | inhibitor | inhibitor | cytotoxicity | Indication |
| ectinascidin 743 | Y | 1 | | 1 | ovarian cancer |
| digitoxin | Y | 2 | None | | cancers, |
| | | | | | congestive heart |
| | | | | | failure |
| ouabain | Y | 3 | None | | hypotension and |
| | | | | | arrhythmias |
| bortezomib | Y | 4 | None | 2 | multiple |
| | | | | | myeloma and |
| | | | | | mantle-cell |
| | | | | | lymphoma |
| chromomycin | Nutraceutical? | 5 | | 3 | CML, testicular |
| A3 | | | | | cancer, Paget's |
| | | | | | disease |
| emetine | Y | | 1 | 4 | protozoan |
| | | | | | infection |
| fluorosalan | Topical | | 2 | | topical |
| | | | | | disinfectant |
| narasin | Not in | | 3 | | coccidiosis |
| | USA | | | | |
| lestaurtinib | Phase I-II | | 4 | 6 | acute myeloid |
| | trial | | | | leukemia |
| tribromsalam | veterinary | | 5 | | liver fluke |
| bithionol | Withdrawn | | 6 | | Photosensitizer. |
| | 1967 | | | | Anthelminthic |
| daunorubicinum | Y | | | 5 | Anti-neoplastic. |
| | | | | | Cancer. |

The interferon regulatory factor 3 (IRF3) and toll like receptor 3 (TLR3), TLR4, and TLR7/8, also have drugs that act as inhibitors. There may be more such drugs among the selective srotonin reuptake inhibitors (SSRIs) and serotonin-norepinephrine reuptake inhibitors (SNRIs.)

TABLE 3
IRF3 - TLR3, TLR4, and TLR7/8 inhibitors.

| | FDA | | | |
|---|---|---|---|---|
| Drug | approved | Drug type | Indication | Cautions |
| sertraline | Y | SSRI | depression, | |
| | | | OCD, anxiety, | |
| | | | PTSD, PMDD | |
| trifluoperazine | Y | phenothiazine | schizophrenia, | dementia, tardive |
| | | | agitation, | dyskinesia, renal |
| | | | chemotherapy | or hepatic impairment |
| fluphenazine | Y | phenothiazine | schizophrenia | dementia, tardive |
| | | | | dyskinesia, leukocyte |
| | | | | suppression |

There is also the keystone cytokine tumor necrosis factor alpha, (TNFα) that can be suppressed. Tumor necrosis factor alpha (TNFα) is the target that has been successful at controlling the inflammation of rheumatoid arthritis. There are several FDA approved TNFα inhibitors, adalimumab, etanercept, and, infliximab. A 5HT2A/2C receptor drug, 2,5-Dimethoxy-4-iodoamphetamine (DOI) is the strongest TNFα inhibitor known although it should not be taken except for short periods because it hits the 5HT2C receptor. Note that these biologics worsen survival in real infections.

The mechanistic target of rapamycin (mTOR) pathway is a further target. Rapamycin and similar drugs will make it possible to knock down the adaptive immune system so that it will not generate antibodies and T-cells specific to the antiviral vector. Therefore, it makes sense to add rapamycin, or another mTOR pathway inhibitor such as, temsirolimus, everolimus, or deforolimus, to the protocol.

### Nucleic Acid Delivery Vehicles

The primary immune stimulation risk is due to the nucleic acid delivery vehicles (NADVs) used to deliver nucleic acids into cells in a living animal, with particular concerns for humans. Commonly these are recombinant versions of existing viruses such as adenovirus (AV), adeno-associated virus (AAV), human immunodeficiency virus (HIV), and the like.

Other (NADVs) exist and are being experimented with. Polyplexes are synthetic peptide sequences, typically dendritic, with high ratios of histidine, arginine and lysine. The polyplex is mixed at greater than 1:1 ratio, typically 4:1 by mass, with DNA and commonly incubated for 45 minutes before injection.

Enveloped polyplexes such as the one described in PCT/US2014/057000 are another NADV. These enclose the polyplex in a silica coating which may have a polymer attached to the outside that has the purpose of targeting specific cell types.

The inventor has discussed the issue of immune system stimulation with inventors of the polyplex and enveloped polyplex, and maintains the position that in a human body, the extraordinary dose of these types of NADVs that is required to execute delivery of nucleic acids into large fractions of human cells will cause the problem already elaborated on.

Other forms of NADV will be invented in the future, and the discussion preceding is not exhaustive. The two alternate NADVs described are currently the best probable methods that the inventor knows of. They are sufficiently good that he considers them reasonable to use.

Synthetic NADVs have, in most cases, been rather toxic, so much so that there is little reason, in their present form, to attempt to use them for the kind of large-scale in-vivo nucleic acid delivery described. Liposomes are an example of this type of synthetic toxic NADV.

### Risk Management

The method of using the drugs and biologics above seeks to reduce or eliminate immune system activation by counteraction with drugs. Consequently, responding to manifestations of synthetic sepsis/multiple organ failure may be an integral part of the disclosed method should drug doses be insufficient. For the remainder of this specification, the syndrome will simply be called sepsis, as for most practical purposes this is sufficient, and it communicates well the danger of a crisis should it occur. Sepsis induction depends on dose of antigen administered, which will be an NADV in the present case, together with liver function status and possible unknown factors. Patients should be carefully monitored and an evaluation should take place prior to delivery of the gene therapy protocol.

Prior to treatment the patient's liver status relative to protein synthesis should be evaluated. The reason is that if a patient has compromised liver function the effective dose can potentially be 1/10,000^{th} of the effective dose in a non-compromised patient. While this is not necessarily a disqualification of the patient, it may require adjustment of dose, the length of time the patient is treated, and possibly use of additional agents.

Prior to beginning the protocol, two, sequential doses of IV endotoxin, first 2.5 ng/kg and second 5 ng/kg should be administered to observe the patient response, 36 hours apart. The patient response may serve as an indicator as to whether the patient may require higher doses, or a longer period of time while medicated.

If there is a problem with this part of the protocol, and a patient hyper-responds, this can be treated with:
a. Administration of IV Polymyxin B sulfate starting at 1 mg/kg and up to 5 mg/kg in 24 hours. The LD₅₀ for this antibiotic is unclear. Rodents tolerate 100 of mg/kg and more. Dogs begin dying at 8 mg/kg. the 5 mg/kg is probably a guideline. Careful monitoring of the patient, with special attention to kidney function may allow higher doses in an emergency situations.
b. Injection or oral administration of DMOG.
c. Injection of IL-10. The basis of this is that IL-10 has shown to be effective in animal studies at tempering sepsis as long as there is not an underlying infection.
d. Injection of ghrelin. Ghrelin has been shown to rescue from sepsis with remarkable effect.
e. Administration of adalimumab, etanercept, infliximab, DOI or some other as TNF-α antagonist. DOI dose is in the range of 100 to 500 micrograms per kg and is not an approved drug.
   f. Administration of Anti-Inflammatory Steroids.

### Immune System Shutdown Risk

It is unlikely, however as a patient is dosed with these drugs, their condition may come to resemble that of a person who is experiencing serious radiation poisoning if the term is protracted. For instance, in the case of a patient with unexpected liver compromise, or if it is necessary to give the patient an unusual amount of time to clear the antigenic material from the gene therapy inoculation, the patient may see a drop in neutrophils, NK cells and T-cells. This may necessitate dosing them with Epogen, ghrelin and/or antibiotics and other drug standards of care for radiation poisoning. Care should be taken to ensure the gut maintains motility to prevent infection. If the patient suffers an infection while in this state, hyperbaric oxygen treatments for 1 hour at 1.6-2.2 bar should be initiated and repeated until the patient is clearly well.

### Response to Anaphylaxis

It is possible that a patient may develop an anaphylactic reaction to some component of the treatment. Standard materials for dealing with this situation are preferably available and on hand. (e.g. Epinephrine, corticosteroids, antihistamine, airway equipment.) As corticosteroids interfere dramatically with immune system response, epinephrine and antihistamine are preferable. Administration of antihistamines as a prophylactic similar to the way it is done in chemotherapy should not interfere with the procedure described here.

### Part 2: Expression of CRISPR Cassette

For purposes of this application, the CRISPR cassette refers to all of the genes that are required for a CRISPR system to be active, or any of the parts to that system. A CRISPR cassette in this usage could include more than one plasmid, or it could be all in one plasmid. In this usage, a plasmid also includes such systems as the one that produces an expression cassette in the primary E. coli genome and uses a topoisomerase or similar enzyme to cut the expression cassette out of the E. coli gene.

Part 2 comprises the use of CpG-ODNs as vehicles or parts of the expression cassette and using a promoter variant such as the tetR or TRE that is activated by the presence of tetracycline or doxycycline. Other switches may be used.

There are two basic types of techniques that will maximize the pulsatile expression of a CRISPR gene therapy and minimize its expression long-term. These are to make use of base plasmids that contain CpG-ODN motifs recognized by mammalian cells, and to control expression using an inducible promoter. This problem of CpG sequences in DNA expression has been recognized as an issue for many years that causes ectopic plasmid gene expression to drop to single digit expression percentages within a few weeks. This part of Part 2 of the specification makes use of this problem as a desirable feature, reversing the CpG-free system to achieve short term expression.

The provision of CpG-ODN motifs may result in atherosclerosis if continued and TLR9 is allowed to express at a significant level, which is a significant concern.

The expression of the CRISPR cassette will be under the control of tetR or some other eukaryotic activated promoter system.

Using a CHYSEL system linker, the expression cassette may have an HDR protein linked to the rest of the expression cassette between the promoter and a termination sequence, generally poly-A.

### Advantageous Effects of Invention

The invention has application to two important areas of clinical therapeutics, CRISPR gene therapy and general gene therapy. Both areas are of high importance to society and improvements in this area can provide significant benefit. Therefore, the foregoing is considered as illustrative only of the principles of the invention. Further, since numerous modifications and changes will readily occur to those skilled in the art, it is not desired to limit the invention to the exact disclosure shown and described, and accordingly, all suitable modifications and equivalents may be resorted to, falling within the scope of the invention.

Part 1 of this specification addresses a major problem in gene therapy delivery, which is that if one attempts to deliver the required dose of a gene therapy for conditions, such as muscular dystrophy that require large fractions of cells to be transfected, this will kill the patient. The present invention solves this problem in that it is made possible to transform most somatic cells that matter. (e.g. cells with nuclei.) Part 2 of this invention improves the controllability and minimizes CRISPR activity when it is no longer desired. This part of the invention also allows for CRISPR activity to be turned back on iteratively where it is desired to titrate the gene therapy's activity.

### INDUSTRIAL APPLICABILITY

The foregoing invention is a physical product that can be manufactured and applied within the field of gene therapy in medicine, and in agricultural practice with livestock.

### DESCRIPTION OF EMBODIMENT

### The Preferred Embodiment of Part 1

The preferred embodiment of Part 1 of the invention is a method comprising delivery of drugs to inhibit the immune system, the method comprising the steps.
a. An inhibitor of NFκB.
b. An inhibitor or IRF3, TLR3, TLR4, and TLR7/8. The SSRI, sertraline, is preferred due to lower toxicity. Other SSRI or SNRI drugs may also prove to work for this purpose.
c. A TLR9 inhibitor if it is required.
d. A TNFα inhibitor.
e. An mTOR inhibitor.
f. An NADV for delivery of nucleic acid into cells of a living animal.

### The Preferred Embodiment of Part 2

The preferred embodiment of Part 2 of the invention is a structure comprising:
a. An expression vector plasmid containing CpG-ODNs.
b. An expression vector plasmid with a promoter dependent on a TetR system.
c. An expression vector plasmid with a promoter, and multiple protein coding sequences with CHYSEL linkers between them.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

The foregoing description of the preferred embodiment of the present invention has been presented for the purpose of illustration and description. It is not intended to be exhaustive or to limit the invention to the precise form disclosed. Many modifications and variations are possible in light of the above teachings. It is intended that the scope of the present invention not be limited by this detailed description, but by the claims and the equivalents to the claims appended hereto.

I claim:

1. A method for delivering nucleic acid sequences into a subject-animal's cells, wherein a subject-animal is either, a human, or a non-human animal modified to have one or more human immune system vulnerabilities, the method comprising:
a. administering to the subject-animal a plurality of immune system modulators comprising:
i. a tumor necrosis factor alpha (TNFα) inhibitor;
ii. a nuclear factor κB (NFκB) inhibitor;
iii. an interferon regulatory factor 3 (IRF3) inhibitor; and
iv. a toll-like receptor 9 (TLR9) inhibitor;
b. delivering said nucleic acid sequences into said subject-animal's cells;
d. wherein said nucleic acid sequences are delivered using a nucleic acid delivery vehicle (NADV); and
e. wherein the dose of NADV is 10¹³ NADV particles or more.

2. The method of claim 1 further comprising the step of administering a mechanistic target of rapamycin (mTOR) inhibitor and wherein said delivering step occurs after each said administering step.

3. The method of claim 2, wherein the mTOR inhibitor is selected from the group consisting of rapamycin, temsirolimus, everolimus, and deforolimus.

4. The method of claim 1 further comprising the step of administering a selective serotonin reuptake inhibitor (SSRI) and wherein said delivering step occurs after each said administering step.

5. The method of claim 4, wherein the SSRI is selected from the group consisting of citalopram, escitalopram, fluoxetine, fluvoxamine, paroxetine, dapoxetine, indalpine, zimelidine, cericlamine, and panuramine.

6. The method of claim 1 further comprising the step of administering a serotonin-norepinephrine reuptake inhibitor (SNRI) and wherein said delivering step occurs after each said administering step.

7. The method of claim 6, wherein the SNRI is selected from the group consisting of venlafaxine, sibutramine, duloxetine, atomoxetine, desvenlafaxine, milnacipran, and levomilnacipran.

8. The method of claim 1, wherein the TNFα inhibitor is selected from the group consisting of adalimumab, etanercept, infliximab, and 2,5-Dimethoxy-4-iodoamphetamine.

9. The method of claim 1, wherein the NFκB inhibitor s selected from the group consisting of ectinascidin 743, digitoxin, ouabain, bortezomib, chromomycin A3, emetine, fluorosalan, narasin, lestaurtinib, tribromsalam, bithionol, and daunorubicinum.

10. The method of claim 1, wherein the IRF3 inhibitor is selected from the group consisting of sertraline, trifluoperazine, and fluphenazine.

11. The method of claim 1, wherein the TLR9 inhibitor is selected from the group consisting of 3-[4-(6-(3-(dimethylamino)propoxy)benzo[d]oxazol-2-yl)phenoxy]-N,N-dimethylpropan-1-amine, 6-[3-(pyrrolidin-1-yl)propoxy)-2-(4-(3-(pyrrolidin-1-yl)propoxy)phenyl]benzo[d]oxazole, and hydroxychloroquine.

12. The method of claim 1 wherein the NADV is a virus capsid.

13. The method of claim 1 wherein the NADV is a polyplex.

14. The method of claim 1 wherein the NADV is an enveloped polyplex.

15. The method of claim 1 wherein said delivering step occurs before said administering step.

16. The method of claim 1 wherein said delivering step occurs simultaneously with said administering step.

17. The method of claim 1 wherein the nucleic acid sequence comprises a nucleic acid sequence containing a nucleic acid sequence coding for clustered regularly interspaced short palindromic repeats (CRISPR) that is under the control of a tetracycline induced promoter.

18. The nucleic acid sequence of claim 17 further comprising cytosine-phosphate-guanine (CpG) oligodeoxynucleotides (CpG-ODN) nucleic acid sequences.

19. The nucleic acid sequence of claim 17 further comprising:
a. one or more cis-acting hydrolase element (CHYSEL) sequences linking two or more gene sequences under the control of said tetracycline induced promoter; and
b. wherein said two or more genes code for proteins.

20. The method of claim 1 wherein said delivering step occurs after said administering step.
